(19) United States
(12) Patent Application Publication
(10) Pub. No.: US 2019/0099155 A1
(43) Pub. Date: Apr. 4, 2019

(54) DIAGNOSTIC IMAGING SYSTEM

(71) Applicant: Shimadzu Corporation, Kyoto (JP)

(72) Inventors: Daisuke NOTOHARA, Kyoto (JP); Tomoharu OKUNO, Kyoto (JP); Shinsuke KANAZAWA, Kyoto (JP); Kazuhiro MORI, Kyoto (JP)

(21) Appl. No.: 16/150,779

(22) Filed: Oct. 3, 2018

(30) Foreign Application Priority Data
Oct. 4, 2017 (JP) 2017-194133

(51) Int. Cl. A61B 8/12; A61B 17/34; A61B 1/06; A61B 8/00

(52) U.S. Cl. CPC A61B 8/12; A61B 17/3478; A61B 2010/045; A61B 8/4444; A61B 1/06

(57) ABSTRACT

This diagnostic imaging system is provided a control unit configured to associate an ultrasonic image capable of identifying a sampling position at the time of sampling a specimen from a subject among ultrasonic images captured by the ultrasonic imaging unit with information for identifying the specimen sampled from the subject.

### CROSS-REFERENCE TO RELATED APPLICATIONS

The present application claims priority to Japanese Patent Application No. JP2017-194133, entitled “diagnostic imaging system”, filed on Oct. 4, 2017, and invented by Daisuke Notohara, Tomoharu Okuno, Shinsuke Kanazawa, and Kazuhiro Mori, the content of which are incorporated herein by reference in their entirety.

### BACKGROUND OF THE INVENTION

### Field of the Invention

The present invention relates to a diagnostic imaging system.

### Description of the Background Art

Conventionally, a diagnosis system equipped with an ultrasonic imaging unit is known. Such a diagnosis system is disclosed in, for example, Japanese Unexamined Patent Application Publication No. 2016-150053.

Japanese Unexamined Patent Application Publication No. 2016-150053 discloses an ultrasonic diagnosis system provided with an ultrasonic endoscope including a puncture needle and an ultrasonic transducer (ultrasonic imaging unit) and a forceps table operation control unit, and a specimen is sampled by a puncture needle. In this ultrasonic diagnosis system, the forceps table operation control unit is configured to control the puncture needle to protrude towards the puncture position based on the puncture position detected in the image captured by the ultrasonic transducer.

### SUMMARY OF THE INVENTION

Here, it is considered that the specimen sampled in the ultrasonic diagnosis system of Japanese Unexamined Patent Application Publication No. 2016-150053 is subjected to a pathological examination using a microscope or the like by a pathologist or the like in order to diagnose the disease, and pathological diagnosis is performed based on the examination result. At that time, for the purpose of preventing the false recognition of the correspondence relation between the examination result of the sampled specimen and the puncture position (sampling position) of the specimen, it is considered that a plurality of doctors will be present at the examination to confirm or a doctor in charge will need to make an effort such as matching the blood sampling position with the analysis result based on the sketch. Therefore, it is considered that there is a problem that the burden on a doctor, etc., involved in handling the sampled specimen is large.

The present invention was made to solve the aforementioned problems, and an object of the present invention is to provide a diagnostic imaging system capable of reducing the burden on a doctor, etc., involved in handling a sampled specimen.

In order to attain the aforementioned object, a diagnostic imaging system according to one aspect of the present invention includes:

an ultrasonic imaging unit configured to capture an ultrasonic image of a subject; and

a control unit configured to associate an ultrasonic image capable of identifying a sampling position at the time of sampling a specimen from the subject among ultrasonic images captured by the ultrasonic imaging unit with information for identifying the specimen sampled from the subject.

In the diagnostic imaging system according to one aspect of the present invention, it becomes possible for a doctor, etc., to identify the sampling position of the specimen associated with the ultrasonic image from the ultrasonic image obtained when a specimen (e.g., a tissue piece) is sampled from a subject. By associating the ultrasonic image at the time of sampling a specimen with the information for identifying the specimen sampled from the subject, for example, when a doctor identifies the sampling position of the specimen from the ultrasonic image, it is possible to easily identify the specimen associated with the identified sampling position. Further, for example, when an examination result of the specimen is obtained, it is possible to associate the sampling position of the specimen with the examination result by the ultrasonic image associated with information for identifying the specimen. As a result, without creating a sketch at the time of sampling the specimen or collating the sampled specimen and the sampling position, the corresponding relation between the sampled specimen and the sampling position (showing the ultrasonic image) can be managed. Therefore, it is possible to reduce the burden on the doctor, etc., involved in handling the sampled specimen.

In the diagnostic imaging system according to the one aspect of the present invention, it is preferable that the information for identifying the specimen include information for identifying the sampling position and the information for identifying the sampling position be associated with the ultrasonic image at the time of sampling the specimen. With this configuration, it is possible to easily grasp the sampling position of the specimen via the ultrasonic image, and it is also possible to suppress the incorrect association between the sampled specimen and the sampling position. As a result, a doctor, etc., can assuredly perform pathological diagnosis, etc., on the position where the specimen was sampled.

In the diagnostic imaging system according to the one aspect of the present invention, it is preferable that the ultrasonic image capable of identifying the sampling position include an ultrasonic image capable of identifying the sampling position by an image of a specimen sampling device arranged at the sampling position of the specimen or near the sampling position of the specimen. With this configuration, since the sampling position can be identified based on the specimen sampling device included in the ultrasonic image, even in cases where a tissue which is difficult to distinguish by an ultrasonic image is sampled as a specimen, e.g., the water content of the tissues are about the same, it is possible to easily identify the sampling position from the position of the specimen sampling device.

In this case, it is preferable that the specimen sampling device include an introduction portion to be introduced into the subject and a sampling portion provided at a tip end portion of the introduction portion and configured to sample the specimen at the sampling position, and the ultrasonic imaging unit include an internal ultrasonic transducer provided at the tip end portion of the introduction portion together with the sampling portion of the specimen sampling device and configured to capture the ultrasonic image of the subject by generating ultrasonic waves in the subject and detecting reflected waves. With such a configuration, even in cases where an ultrasonic image cannot be obtained with sufficient clarity from the outside due to a portion where air is stored, such as, e.g., a stomach where an ultrasonic image is hardly propagated, subcutaneous fat, etc., an ultrasonic image can be obtained from the inside of the subject. Therefore, it is possible to clearly obtain an ultrasonic image at a position where it is difficult to image with sufficient sharpness from the outside. As a result, it is possible to obtain an ultrasonic image capable of clearly identifying the sampling position, which makes it possible to easily identify the sampling position of the specimen from the ultrasonic image. Furthermore, even if it is difficult to identify the sampling position by a sketch of a doctor, etc., due to the fact that a specimen is sampled at an inside of the subject which cannot be directly recognized, based on the ultrasonic image captured by the internal ultrasonic transducer, it is possible to easily identify the sampling position.

In the configuration in which the ultrasonic imaging unit includes the internal ultrasonic transducer, it is preferable that the sampling portion be arranged in the vicinity of the internal ultrasonic transducer and be arranged to be displayed in the ultrasonic image captured by the internal ultrasonic transducer. With this configuration, when the sampling position is identified based on the image of the sampling portion included in the ultrasonic image, it is possible to easily identify the sampling position from the position of the sampling portion. Also, by arranging the sampling portion near the internal ultrasonic transducer, in the ultrasonic image that becomes unclear as the distance from the internal ultrasonic transducer increases, it is possible to clearly display the sampling portion which can be used as a specific index of the sampling position.

In the configuration in which the sampling position can be identified from the image of the specimen sampling device, it is preferable that the ultrasonic imaging unit include an external ultrasonic transducer configured to capture the ultrasonic image of the subject by generating ultrasonic waves from an outside of the subject and detecting reflected waves, and when the specimen at the sampling position is sampled from the outside of the subject, the specimen sampling device be imaged in the ultrasonic image by the external ultrasonic transducer. With this configuration, when the sampling position is identified based on the image of the specimen sampling device included in the ultrasonic image, it is possible to easily identify the sampling position from the position of the sampling portion. Moreover, in the ultrasonic image captured from the outside of the subject by the external ultrasonic transducer, unlike the case of using an ultrasonic image by an ultrasonic transducer in which the imaging position cannot be visually recognized directly due to being placed inside the subject, the external ultrasonic transducer is placed outside the subject, and therefore it is possible to directly visually recognize the external ultrasonic transducer. For this reason, it is possible to easily identify the imaging position in the subject. As a result, it is possible to accurately identify the sampling position in the subject.

In the diagnostic imaging system according to the one aspect of the present invention, it is preferable that the information for identifying the specimen include information for identifying the sampling position and identification information assigned for each specimen at the time of sampling. With this configuration, for each different specimen, it is possible to assuredly associate the ultrasonic image capable of identifying the sampling position with the information for identifying the sampling position via the identification information. Further, by using the identification information, the information for identifying the sampling position and the ultrasonic image capable of identifying the sampling position can be easily managed.

In the diagnostic imaging system according to the one aspect of the present invention, it is preferable that the information for identifying the specimen include information for identifying the sampling position and that the control unit be configured to further associate information for identifying the subject with each of a plurality of ultrasonic images associated with the information for identifying the sampling position. With this configuration, by sampling specimens more than once on the same subject, even when the association between the information for identifying the sampling position and the ultrasonic image is performed a plurality of times, based on the information of the subject, information for identifying the sampling position and the ultrasonic image can be managed collectively. As a result, the information for identifying the sampling position and a plurality of ultrasonic images can be collectively provided as a doctor, etc., as information on the subject.

In the diagnostic imaging system according to the one aspect of the present invention, it is preferable that the information for identifying the specimen include information for identifying the sampling position and an examination result of the specimen sampled at the sampling position. With this configuration, it becomes possible to collectively manage the ultrasonic image capable of identifying the sampling position, the information for identifying the sampling position, and the examination result of the specimen obtained from the sampling position. As a result, it becomes possible to further reduce the burden of a doctor, etc., involved in handling the sampled specimen.

### BRIEF DESCRIPTION OF THE DRAWINGS

FIG. 1 is a schematic diagram showing the overall configuration of a diagnostic imaging system according to a first embodiment.

FIG. 2 is a schematic diagram showing a configuration of an ultrasonic endoscope apparatus according to the first embodiment.

FIG. 3 is an enlarged perspective view showing a tip end portion of a cable of an ultrasonic endoscope.

FIG. 4 is a diagram for explaining the association among identification information, an ultrasonic image, and an examination result.

FIG. 5 is a diagram for explaining the association among subject information, an ultrasonic image, and a sample identifying information according to the first embodiment.

FIG. 6 is a flowchart for explaining association processing according to the first embodiment.

FIG. 7 is a schematic diagram showing the overall configuration of the diagnostic imaging system according to the first embodiment.

FIG. 8 is a schematic diagram showing a configuration of an ultrasonic image device according to the second embodiment.

FIG. 9 is a block diagram showing a configuration of a workstation.

FIG. 10 is a diagram showing an ultrasonic image according to a second embodiment.

### DESCRIPTION OF PREFERRED EMBODIMENTS

Hereinafter, embodiments of the present invention will be described with reference to the drawings.

### First Embodiment

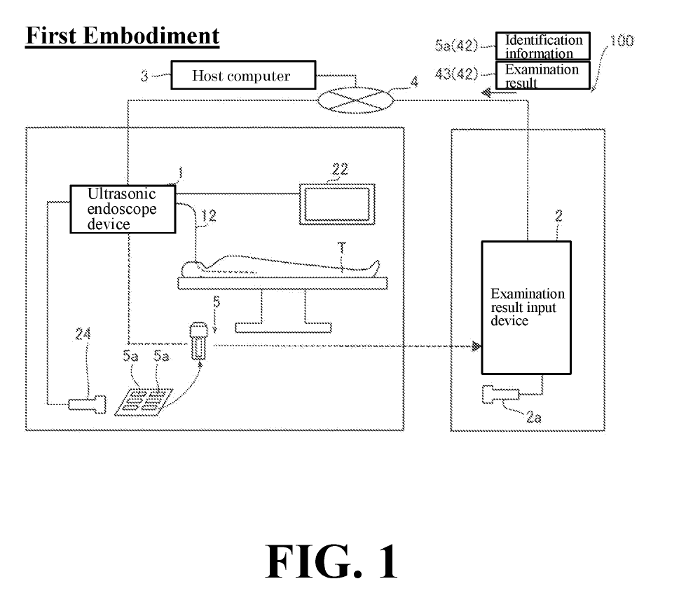

With reference to FIG. 1 to FIG. 5, the configuration of the diagnostic imaging system 100 according to the first embodiment of the present invention will be described.

As shown in FIG. 1, the diagnostic imaging system 100 is a system for associating an ultrasonic image 40 capable of identifying a sampling position P when a specimen 90 is sampled from a subject T and information for identifying a specimen 90 (hereinafter referred to as “specimen identification information 42”). That is, the diagnostic imaging system 100 is configured to associate the specimen 90 sampled from the subject T with the ultrasonic image 40 in which the sampling position P of the specimen 90 is distinguishably displayed by the specimen identification information 42.

The subject T is a subject to be subjected to a diagnosis of diseases, and the specimen 90 for diagnosis is sampled from the subject T by a doctor, etc. The subject T includes humans and other animals.

The specimen 90 includes all biological specimens to be sampled from the subject T, and is not particularly limited. The specimen 90 is, for example, a part or all of a body fluid, such as, e.g., blood or tissue fluid and organs, such as, e.g., internal organs and bones.

Further, the examination result 43 of the specimen 90 includes, for example, pathological diagnosis results for the specimen 90 using, for example, a microscope. When a definitive diagnosis is made by a doctor based on the pathological diagnosis result, it becomes important to identify the lesion. The sampling position P of the specimen 90 is important information for identifying the lesion in conjunction with the component analysis result of the specimen 90 and the pathological diagnosis result to prevent the mix-up of the specimen 90.

Therefore, in the first embodiment, the diagnostic imaging system 100 is provided with an ultrasonic imaging unit 50 configured to capture the ultrasonic image 40 of the subject T and a control unit 60 configured to associate the ultrasonic image 40 capable of identifying the sampling position P with the specimen identification information 42. Hereinafter, the specific configuration of the diagnostic imaging system 100 will be described.

The diagnostic imaging system 100 of the first embodiment includes the ultrasonic endoscope device 1, the examination result input device 2, and the host computer 3. The ultrasonic endoscope device 1, the examination result input device 2, and the host computer 3 are arranged in different rooms or buildings and are electrically connected to each other via the network 4.

### (Configuration of Ultrasonic Endoscope Device)

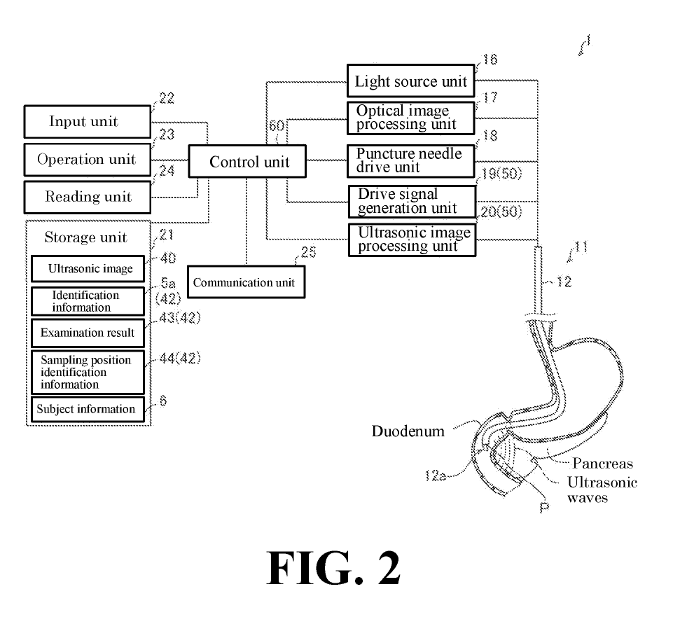

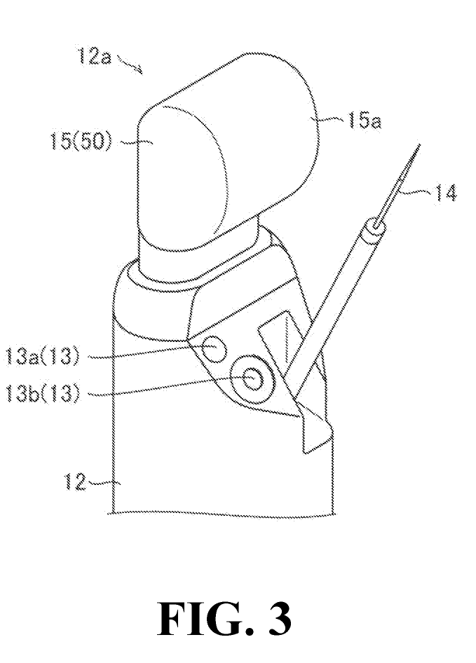

As shown in FIG. 2 and FIG. 3, the ultrasonic endoscope device 1 is provided with an ultrasonic endoscope 11 capable of capturing the ultrasonic image 40 from the inside of the subject T. The ultrasonic endoscope device 1 is further provided with a control unit 60, a light source unit 16 electrically connected to the ultrasonic endoscope 11 and the control unit 60, an optical image processing unit 17, a puncture needle driving unit 18, a drive signal generation unit 19, and an ultrasonic image processing unit 20. The ultrasonic endoscope device 1 is further provided with a storage unit 21, a display unit 22, an operation unit 23, a reading unit 24, and a communication unit 25 which are electrically connected to the control unit 60. Note that the ultrasonic endoscope 11 is an example of the “specimen sampling device” recited in claims.

The ultrasonic endoscope 11 is provided with a cable 12 to be introduced to the inside of the subject T (the inside of the body), and an optical component 13, a puncture needle 14, and an ultrasonic transducer 15 which are provided at the tip end portion 12a of the cable 12 so as to be introduced into the inside (the inside of the subject) of the subject T. Note that the cable 12 and the puncture needle 14 are examples of the “introduction portion” and the “sampling portion” recited in claims, respectively.

In the first embodiment, an example will be described in which a tissue piece of a pancreas is sampled as a specimen 90 in a state in which the tip end portion 12a of the cable 12 is arranged in a duodenum inside the subject T.

The cable 12 is composed of a hollow tube, and will be introduced into the subject T via the oral route or the like. Within the cable 12, an optical cable (not shown) which guides the light from the light source unit 16 to the optical parts 13 and also guides the light (reflected light) from the optical parts 13 to the optical image processing unit 17, a signal line (not shown) that transmits a drive signal from the drive signal generation unit 19 to the ultrasonic transducer 15 and also transmits a detection signal from the ultrasonic transducer 15 to the ultrasonic image processing unit 20, an operating line (not shown) for bending the cable 12 and protruding the puncture needle 14, etc., are arranged.

The optical parts 13 have a light guide 13a for irradiating light to the inside of the subject T from the tip end portion 12a of the cable 12 and a light receiving portion 13b for receiving the reflected light reflected in the subject T at the tip end portion 12a. Then, based on the reflected light received by the light receiving portion 13b, an image (not shown) is created in the optical image processing unit 17. With this, a doctor, etc., can make the tip end portion 12a of the cable 12 reach a predetermined position in the duodenum inside the subject T while confirming the image displayed on the display unit 22. Hereinafter, the image obtained through the optical parts 13 is referred to as an “optical image”, and is distinguished from the ultrasonic image 40 obtained through the ultrasonic transducer 15.

The puncture needle 14 is configured to be stored in the tip end portion 12a of the cable 12. The puncture needle 14 is configured so that a specimen 90 can be sampled by sucking the specimen 90 into the inside of the needle at the sampling position P of the specimen 90 in the subject T in a projected state.

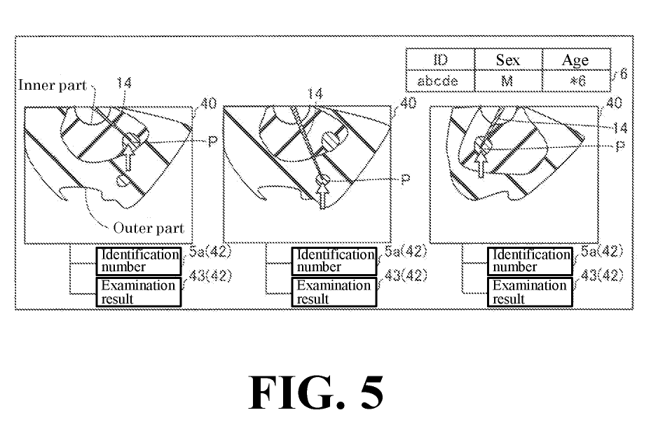

The ultrasonic transducer 15 is provided at the tip end portion 12a of the cable 12, and is configured to generate ultrasonic waves when a drive signal is applied to the signal line and output a detection signal to the signal line in response to reflected waves of the ultrasonic waves. Based on the detection signal, a two-dimensional ultrasonic image 40 (see FIG. 5) is created in the ultrasonic image processing unit 20 and displayed on the display unit 22. Here, as the ultrasonic image 40, an ultrasonic image 40 spread in a radial shape (in a fan-shape) is obtained. The inner part of the fan-shaped ultrasonic image 40 is an image of the subject T in the vicinity of the ultrasonic transducer 15 (duodenum), and the outer part of the fan-shaped ultrasonic image 40 is the image of the subject T at a position away from the ultrasonic transducer 15.

The ultrasonic image 40 is preferably displayed in a so-called B (Brightness) mode display in which the intensity of the ultrasonic waves corresponds to the magnitude of the luminance. Further, the ultrasonic transducer 15 is of a convex type which radially emits ultrasonic waves, and an ultrasonic wave emitting surface 15a is formed in an arc shape. Note that the ultrasonic transducer 15 is not limited to the convex type, and may be, for example, a linear type.

The puncture needle 14 is provided at the tip end portion 12a of the cable 12 together with the ultrasonic transducer 15, so that it is arranged in the vicinity of the ultrasonic transducer 15. In addition, the puncture needle 14 is arranged so as to be displayed in the ultrasonic image 40 captured by the ultrasonic transducer 15. Specifically, the puncture needle 14 is configured to protrude from the tip end portion 12a of the cable 12 so as to face the ultrasonic wave emitting surface 15a of the ultrasonic transducer 15 provided at the tip end portion 12a of the cable 12. With this, the puncture needle 14 is displayed in the ultrasonic image 40 when it is protruded to a position where it reflects ultrasonic waves from the ultrasonic transducer 15.

The specimen 90 sampled from the sampling position P by the puncture needle 14 is accommodated in the specimen container 5 in which a label showing identification information 5a different for each specimen 90 is allotted to the specimen container 5. As a result, corresponding identification information 5a is associated with each specimen 90 one by one. Then, the specimen 90 is transferred to a room where the examination result input device 2 is placed and the pathological diagnosis is performed by a pathologist, etc. Note that the identification information 5a is, for example, a specimen ID attached in the form of a bar code or a two-dimensional code. Note that the identification information 5a is an example of the “information for identifying a specimen” recited in claims.

As shown in FIG. 2, the control unit 60 is a computer configured by including a CPU, a ROM, a RAM, and the like. The control unit 60 functions as a control unit for controlling each part of the ultrasonic endoscope device 1 by executing a predetermined control program by the CPU. Further, the control unit 60 has a function of associating the ultrasonic image 40 capable of identifying the sampling position P with the specimen identification information 42. The details of the association function of the control unit 60 will be described later.

The light source unit 16 has a function of supplying visible light or the like to an optical cable (not shown) under the control of the control unit 60. The optical image processing unit 17 has a function of creating an optical image in a moving image format on the basis of the reflected light received in the optical parts 13 (light receiving portion 13b). The puncture needle driving unit 18 has a function of switching the state of the puncture needle 14 between a storage state and a protruding state under the control of the control unit 60.

The drive signal generation unit 19 has a function of generating an ultrasonic wave drive signal for making the ultrasonic transducer 15 generate ultrasonic waves and transmitting the generated signal to a signal line (not shown) under the control of the control unit 60. The ultrasonic image processing unit 20 has a function of creating an ultrasonic image in a moving image format based on the detection signal from the ultrasonic transducer 15. The ultrasonic imaging unit 50 is composed of the ultrasonic transducer 15, the drive signal generation unit 19, and the ultrasonic image processing unit 20.

The storage unit 21 is configured to store the data of the ultrasonic image 40, the data of the specimen identification information 42, the data of the examination result 43 of the specimen 90, and the like. The display unit 22 is, for example, a monitor, such as, e.g., a liquid crystal display, and has a function of displaying the optical image and the ultrasonic image 40. The operation unit 23 is configured by including, for example, a keyboard and a mouse, a touch panel, another controller, etc., and an input operation or the like to the ultrasonic endoscope device 1 is performed by a doctor, etc.

The reading unit 24 has a function of reading the identification information 5a allotted to the specimen container 5. The communication unit 25 is configured to communicate with the examination result input device 2 and the host computer 3 via the network 4. The ultrasonic endoscope device 1 transmits the data of the ultrasonic image 40, the data of the specimen identification information 42, the data of examination result 43 of the specimen 90, etc., to the host computer 3 via the network 4.

### (Configuration of Examination Result Input Device)

The examination result input device 2 is a device to which the examination result 43 of the specimen 90 (tissue piece) is input by a pathologist, etc., as shown in FIG. 1. The examination result input device 2 is provided with a reading unit 2a for reading the identification information 5a allotted to the specimen container 5. The examination result input device 2 is configured to transmit the identification information 5a read by the reading unit 2a and the input examination result 43 to the ultrasonic endoscope device 1 in a state of being associated with each other.

### (Configuration of Host Computer)

The host computer 3 is a so-called server. The host computer 3 receives the data of the ultrasonic image 40, the data of the specimen identification information 42, etc., from the ultrasonic endoscope device 1 via the network 4 and stores it via the network 4. In the host computer 3, the subject information 6 is stored. Note that, as the subject information 6, there is a patient ID assigned to the subject T. As the subject information 6, the sex of the subject T and the age of the subject T may be used.

### (Association of Ultrasonic Image and Specimen Identification Information)

In the first embodiment, as described above, the control unit 60 is configured to associate the specimen 90 sampled from the subject T with the ultrasonic image 40 in which the sampling position P of the specimen 90 is distinguishably displayed by the specimen identification information 42. Hereinafter, concrete associations will be explained.

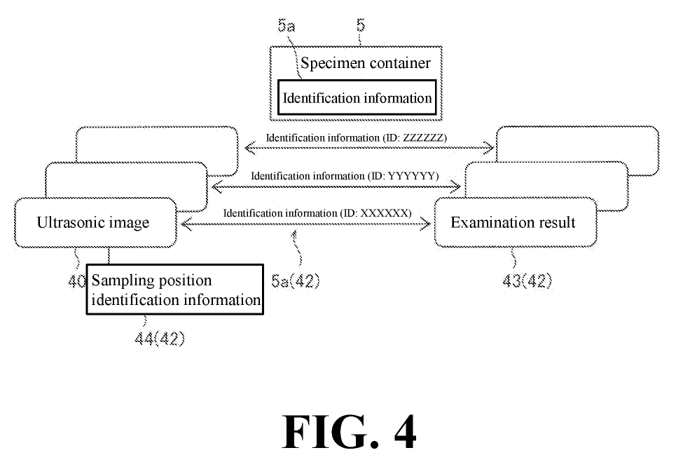

The control unit 60 acquires the data of the identification information 5a (see FIG. 4) and the data of the examination result 43 of the specimen 90 from the examination result input device 2 via the communication unit 25. Further, the control unit 60 acquires the ultrasonic image 40 capable of identifying the sampling position P from the storage unit 21.

Here, the ultrasonic image 40 (see FIG. 5) capable of identifying the sampling position P is obtained by imaging (photographing) the region including the sampling position P in a visually recognizable manner when sampling the specimen 90 identified by the specimen identification information 42. Here, the ultrasonic transducer 15 of the ultrasonic endoscope 11 is particularly effective when imaging a gall bladder, a pancreas, etc., which is impossible to be optically imaged since the cable 12 of the ultrasonic endoscope 11 cannot be reached due to the fact that the gall bladder, the pancreas, etc., is positioned ahead of a tube (bile duct, etc.) having a tubular diameter smaller than the cable 12.

In the ultrasonic image 40, the image of the puncture needle 14 is displayed in the ultrasonic image 40. With this, in the ultrasonic image 40 at the time of sampling the specimen 90, it is possible to set the tip of the puncture needle 14 to the sampling position P of the specimen 90.

Further, as shown in FIG. 5, it is preferable to associate the sampling position identifying information 44 (sampling position identifying information) with the ultrasonic image 40 by identifying the sampling position P on the ultrasonic image 40 by specifically indicating the sampling position P on the ultrasonic image 40 (by the hollow arrow in FIG. 5). In this case, a doctor, etc., can identify the sampling position P on the ultrasonic image 40 by performing the operation of identifying the sampling position P on the ultrasonic image 40 by using the operation unit 23.

Alternatively, it may be possible to associate the sampling position identifying information 44 with the identifiable ultrasonic image 40 capable of identifying the sampling position P by associating (linking) the information of the sampling position P with the ultrasonic image 40 without identifying the sampling position P on the ultrasonic image 40.

By capturing an X-ray image using X-rays in a state in which the tip end portion 12a of the cable 12 is located at the imaging position when sampling the specimen 90, it is possible to accurately recognize the position of the ultrasonic transducer 15 and the puncture needle 14 within the subject T when the ultrasonic image 40 is captured. In this case, it is possible to associate the X-ray image with the ultrasonic image 40 capable of identifying the sampling position P as the sampling position identifying information 44. Also, based on the length of the cable 12 introduced in the subject T, it is possible to estimate the position of the ultrasonic transducer 15 and the puncture needle 14 in the subject T when the ultrasonic image 40 is captured. In this case, it is possible to associate the length of the cable 12 with the ultrasonic image 40 capable of identifying the sampling position P as the sampling position identifying information 44. In these cases, even in cases where the puncture needle 14 is not displayed in the ultrasonic image 40, the sampling position P at the time of sampling the specimen 90 can be indirectly identified. Therefore, the information for identifying the specimen 90 sampled from the subject T may be associated with the ultrasonic image 40 with which the X-ray image or the length of the cable 12 is associated.

Then, as shown in FIG. 4, the control unit 60 associates the specimen identification information 42 with the ultrasonic image 40 capable of identifying the sampling position P. That is, the control unit 60 associates the sampling position identifying information 44 and the examination result 43 of the specimen 90 with the ultrasonic image 40 capable of identifying the sampling position P via the identification information 5a. Note that the specimen identification information 42 related to different specimens 90 may be associated with the same ultrasonic image 40. Then, the identification information 5a, the sampling position identifying information 44, the examination result 43 of the specimen 90, and the ultrasonic image 40 capable of identifying the sampling position P, which are associated with each other, are transmitted to the host computer 3 via the communication unit 25 and stored. With this, a doctor, etc., can confirm in a state in which the identification information 5a, the sampling position identifying information 44, the examination result 43 of the specimen 90, and the ultrasonic image 40 capable of identifying the sampling position P are associated with each other, by accessing the host computer 3 via a terminal (not shown).

Further, as shown in FIG. 5, the control unit 60 associates the information (subject information) 6 for identifying the subject with a plurality of ultrasonic images 40. For example, at the time of sampling the specimen 90, the ID of the subject T is input by a doctor, etc., and sent to the host computer 3. With this, the control unit 60 acquires the subject information 6 from the host computer 3 and associates the identification information 5a with the subject information 6 for each sampling. As a result, it is possible to associate the subject information 6 with the ultrasonic image 40 associated with each of the plurality of identification information 5a. Thus, it is possible to collectively manage the information on the plurality of specimens 90 of the subject T.

### (Association Process)

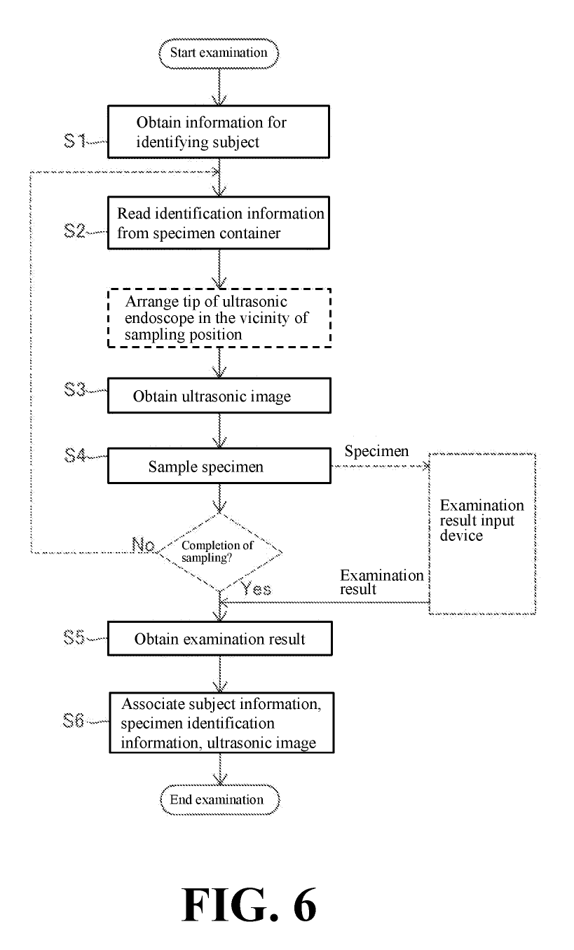

Next, with reference to FIG. 6, the flow of the process of associating the ultrasonic image 40 with the specimen identification information 42 by the diagnostic imaging system 100 (the control unit 60 of the ultrasonic endoscope device 1) will be described.

When the examination is started, first, in Step S1, the control unit 60 acquires the subject information 6 and stores it in the storage unit 21. Then, the control unit 60 acquires the optical image inside the subject T by using the optical parts 13, the light source unit 16, and the optical image processing unit 17 and displays it on the display unit 22. Thereafter, in Step S 2, the control unit 60 reads the identification information 5a from the specimen container 5 based on the reading operation by a doctor via the reading unit 24 and associates it with the subject information 6.

Then, using the optical image displayed on the display unit 22, a doctor introduces the ultrasonic endoscope 11 into the subject T and sends the tip end portion 12a of the cable 12 to the vicinity (duodenum) of the sampling position P.

In Step S3, the control unit 60 acquires the ultrasonic image 40 on which the sampling position P of the subject T is displayed, using the ultrasonic transducer 15, the drive signal generation unit 19, and the ultrasonic image processing unit 20 based on the ultrasonic image acquisition operation by the doctor, and displays it on the display unit 22. In Step S4, the control unit 60 samples the specimen 90 of the sampling position P using the puncture needle 14 and the puncture needle driving unit 18 based on the specimen acquisition operation by the doctor. Then, the specimen 90 is accommodated in the specimen container 5, and the specimen 90 is associated with the identification information 5a. The process of Steps S2 to S4 concerning the sampling of this specimen 90 is performed every sampling.

After that, the sampled specimen 90 is pathologically diagnosed by a pathologist and the examination result 43 is input to the examination result input device 2. Then, the identification information 5a and the examination result 43 are transmitted to the ultrasonic endoscope device 1 from the examination result input device 2 in a state of being associated with each other.

In Step S5, the control unit 60 acquires the examination result 43 associated with the identification information 5a. In Step S6, the control unit 60 associates the subject information 6, the specimen identification information 42 (identification information 5a, examination result 43, sampling position identifying information 44), and the ultrasonic image 40 capable of identifying the sampling position P. Thereafter, the control unit 60 transmits the subject information 6, the specimen identification information 42, and the ultrasonic image 40 capable of identifying the sampling position P, which are associated with each other, to the host computer 3. With this, the examination is completed.

### (Effects of First Embodiment)

In the first embodiment, the following effects can be obtained.

In the first embodiment, as described above, the ultrasonic image 40 capable of identifying the sampling position P at the time of sampling the specimen 90 from the subject T is associated with the specimen identification information 42 sampled from the subject T. As a result, a doctor, etc., can identify the sampling position P of the specimen 90 associated with the ultrasonic image 40 captured when the specimen 90 (pancreatic tissue piece) is sampled from the subject T. By associating the ultrasonic image 40 at the time of sampling the specimen 90 with the specimen identification information 42 sampled from the subject T, when the doctor identifies the sampling position P of the specimen 90 from the ultrasonic image 40, it is possible to easily identify the specimen 90 associated with the identified sampling position P. Further, for example, when the examination result 43 of the specimen 90 is obtained, the sampling position P of the specimen 90 and the examination result 43 can be associated with each other by the ultrasonic image 40 associated with the specimen identification information 42. As a result, without creating a sketch at the time of sampling the specimen or collating the sampled specimen with the examination result 43 of the specimen 90, the corresponding relation between the sampled specimen 90 and the sampling position P (showing the the ultrasonic image 40) can be managed. Therefore, it is possible to reduce the burden of a doctor, etc., involved in handling the sampled specimen 90.

Further, in the first embodiment, as described above, the sampling position identifying information 44 is associated with the ultrasonic image 40 at the time of sampling the specimen 90. With this, it is possible to easily grasp the sampling position P of the specimen 90 via the ultrasonic image 40, and it is also possible to suppress the incorrect association between the sampled specimen 90 and the sampling position P. As a result, a doctor, etc., can assuredly perform pathological diagnosis, etc., on the position where the specimen 90 was sampled.

Further, in the first embodiment, as described above, the ultrasonic image 40 capable of identifying the sampling position P is the ultrasonic image 40 capable of identifying the sampling position P of the specimen 90. As a result, since the sampling position P can be identified based on the specimen sampling device included in the ultrasonic image 40, even in cases where a tissue that is difficult to be distinguished by the ultrasonic image 40 due to the reason that the water content of the tissue is about the same is sampled as a specimen 90, it is possible to easily identify the sampling position P from the position of the ultrasonic endoscope 11.

Further, in the first embodiment, as described above, the ultrasonic transducer 15 is provided at the tip end portion 12a of the cable 12 together with the puncture needle 14 of the ultrasonic endoscope 11, generates ultrasonic waves in the subject T, and detects the reflected waves to thereby capture the ultrasonic image 40 of the subject T. With this, even in cases where an ultrasonic image 40 cannot be obtained with sufficient sharpness from the outside due to a portion where air is retained, such as, e.g., a stomach, or subcutaneous fat, etc., where ultrasonic waves are less likely to propagate, the ultrasonic image 40 can be obtained from the inside of the subject T. Therefore, it is possible to clearly obtain the ultrasonic image 40 at a position where it is difficult to image with sufficient sharpness from the outside. As a result, it is possible to capture the ultrasonic image 40 capable of clearly identifying the sampling position P, which makes it possible to easily identify the sampling position P of the specimen 90 from the ultrasonic image 40. Furthermore, even if it is difficult to identify the sampling position P by a sketch by a doctor, etc., due to the fact that the specimen 90 is sampled at an inside of the subject T which cannot be directly visually recognized, based on the ultrasonic image 40 captured by the internal ultrasonic transducer, it is possible to easily identify the sampling position P.

In the first embodiment, as described above, the puncture needle 14 is arranged in the vicinity of the ultrasonic transducer 15 and arranged so as to be displayed in the ultrasonic image 40 captured by the ultrasonic transducer 15. Accordingly, when identifying the sampling position P based on the image of the puncture needle 14 included in the ultrasonic image 40, it is possible to easily identify the sampling position P from the position of the puncture needle 14. In addition, in the ultrasonic image 40 which becomes unclear as the distance from the internal ultrasonic transducer increases by arranging the puncture needle 14 in the vicinity of the ultrasonic image 40, it is possible to clearly display the puncture needle 14 which can be used as a specific index of the sampling position P.

Further, in the first embodiment, as described above, the specimen identification information 42 includes the sampling position identifying information 44 and the identification information 5a assigned to each specimen 90 at the time of sampling. With this, for each different specimen 90, it is possible to assuredly associate the ultrasonic image 40 capable of identifying the sampling position P with the sampling position identifying information 44 via the identification information 5a. Further, with the identification information 5a, it is possible to easily manage the sampling position identifying information 44 and the ultrasonic image 40 capable of identifying the sampling position P.

In the first embodiment, as described above, the control unit 60 associates the subject information 6 for identifying the subject T with each of the plurality of ultrasonic images 40 associated with the sampling position identifying information 44. As a result, even in cases where the association between the sampling position identifying information 44 and the ultrasonic image 40 is performed plural times by performing the sampling of the specimen 90 plural times for the same subject T, based on the information of the subject T, it is possible to collectively manage the sampling position identifying information 44 and the ultrasonic image 40. As a result, as the information on the subject T, it is possible to collectively provide the sampling position identifying information 44, and the plurality of ultrasonic images 40 to a doctor, etc.

Further, in the first embodiment, as described above, the specimen identification information 42 includes the sampling position identifying information 44 and the examination result 43 of the specimen 90 sampled at the sampling position P. With this configuration, it becomes possible to collectively manage the ultrasonic image 40 capable of identifying the sampling position P, the sampling position identifying information 44, and the examination result 43 of the specimen 90 sampled from the sampling position P. As a result, it becomes possible to further reduce the burden of a doctor, etc., involved in handling the sampled specimen 90.

### Second Embodiment

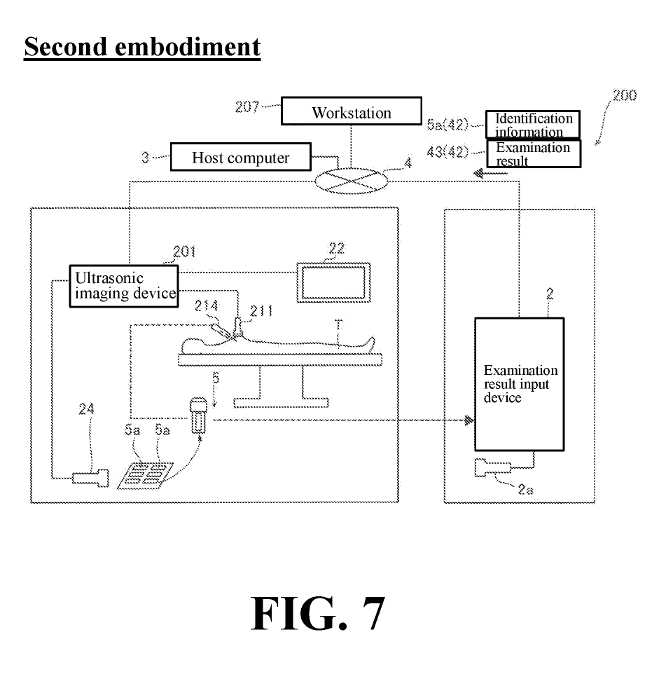

Next, with reference to FIG. 7 to FIG. 10, the configuration of the diagnostic imaging system 200 according to a second embodiment of the present invention will be described. In the second embodiment, the description is directed to the case in which the ultrasonic image 240 is captured from the outside (in vitro) of the subject T and the workstation 207 associates the specimen identification information 42 with the ultrasonic image 240. In the second embodiment, an example in which a tissue piece of a breast of a subject T is obtained as a specimen 90 will be explained. The same reference numerals are allotted to the same configurations as those of the first embodiment, and the description thereof will be omitted.

The diagnostic imaging system 200 of the second embodiment is provided with an ultrasonic imaging device 201, an examination result input device 2, a host computer 3, and a workstation 207.

### (Configuration of Ultrasonic Imaging Device)

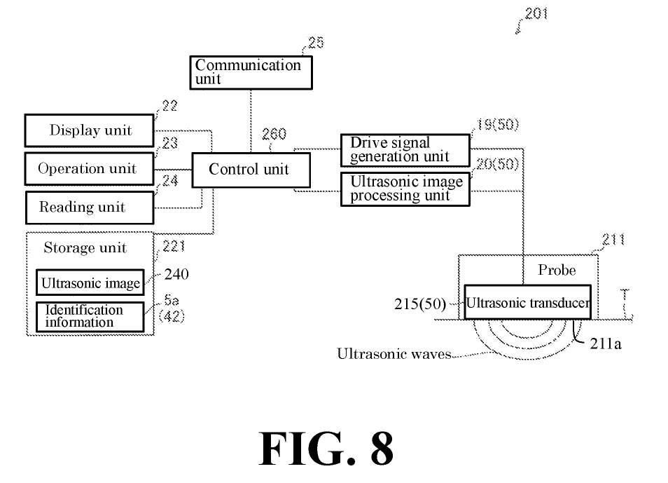

As shown in FIGS. 7 and 8, the ultrasonic imaging device 201 is provided with an ultrasonic probe 211 capable of imaging an ultrasonic image 240 from the outside of the subject T. Further, the ultrasonic imaging device 201 is further provided with a control unit 260, and a drive signal generation unit 19 and an ultrasonic image processing unit 20 which are electrically connected to the ultrasonic probe 211 and the control unit 260. The ultrasonic imaging device 201 is further provided with a storage unit 221, a display unit 22, an operation unit 23, a reading unit 24, and a communication unit 25.

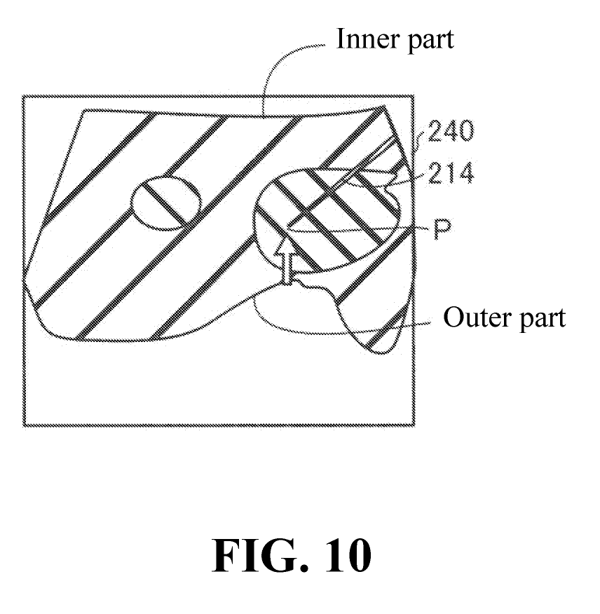

The ultrasonic probe 211 is configured to be grasped by a doctor, etc. In addition, an ultrasonic transducer 215 is arranged in the vicinity of the contact surface 211 a with the subject T of the ultrasonic probe 211. With this, the ultrasonic transducer 215 generates ultrasonic waves when a drive signal is applied to the signal line in a state in which the ultrasonic probe 211 is in contact with the subject T (breast). The ultrasonic transducer 215 is configured to receive reflected waves of the ultrasonic waves and output a detection signal to the signal line. Based on the detection signal, a two-dimensional ultrasonic image 240 (see FIG. 10) is created in the ultrasonic image processing unit 20 and displayed on the display unit 22. Here, as the ultrasonic image 240, an ultrasonic image 240 spread in a slightly radial shape (in a fan-shape) is obtained. The inner part of the fan-shaped ultrasonic image 240 is an image of the subject T in the vicinity of the ultrasonic transducer 215 (outside of the subject T), and the outer part of the fan-shaped ultrasonic image 240 is an image of the subject T at a position away from the ultrasonic transducer 215. The ultrasonic transducer 215, the drive signal generation unit 19, and the ultrasonic image processing unit 20 constitute an ultrasonic imaging unit 250. Note that the ultrasonic transducer 215 is an example of the “external ultrasonic transducer” recited in claims.

Further, the ultrasonic imaging device 201 is configured to be able to capture an ultrasonic image 240 (see FIG. 10) including a puncture needle 214 for sampling a specimen 90 from the outside of the subject T. With this, in the ultrasonic image 240 at the time of sampling the specimen 90, it is possible to set the tip of the puncture needle 214 to the sampling position P of the specimen 90.

Note that the puncture needle 14 is configured so that a specimen 90 can be sampled by sucking the specimen 90 into the inside of the needle at the sampling position P of the specimen 90 in the subject T. Then the specimen 90 sampled from the sampling position P by the puncture needle 214 is accommodated in the specimen container 5. Note that the puncture needle 214 is an example of the “specimen sampling device” recited in claims.

The control unit 260 is a computer configured by including a CPU, a ROM, a RAM, and the like. The control unit 260 functions as a control unit for controlling each part of the ultrasonic imaging device 201 by executing a predetermined control program by the CPU. Note that, unlike the first embodiment, the control unit 260 does not function as an associating unit that associates the ultrasonic image 240 capable of identifying the sampling position P with the specimen identification information 42.

The storage unit 221 is configured to store the data of the ultrasonic image 240, the data of the identification information 5a, and the like.

### (Configuration of Workstation)

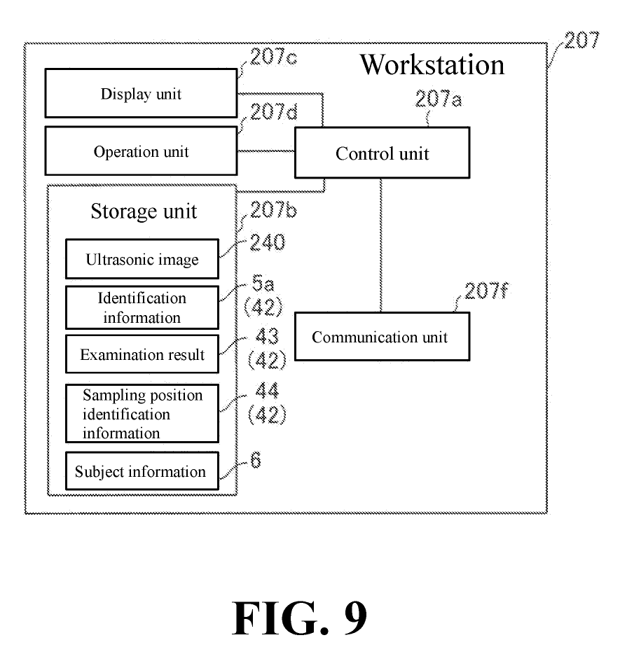

As shown in FIG. 9, the workstation 207 includes a control unit 207a having a function of associating the ultrasonic image 240 capable of identifying the sampling position P with the specimen identification information 42. Further, the workstation 207 is provided with a storage unit 207b, a display unit 207c, an operation unit 207d, a reading unit 207e, and a communication unit 207f.

The storage unit 207b is configured to store the data of the ultrasonic image 240, the data of the specimen identification information 42, the data of the examination result 43 of the specimen 90, and the like. The display unit 207c is, for example, a monitor, such as, e.g., a liquid crystal display, and has a function of displaying the ultrasonic image 240. The operation unit 207d is configured by including, for example, a keyboard and a mouse, a touch panel, another controller, etc., and an input operation or the like to the workstation 207 is performed by a doctor, etc.

The reading unit 207e has a function of reading the identification information 5a allotted to the specimen container 5. The communication unit 25 is configured to communicate with the examination result input device 2 and the host computer 3 via the network 4. The workstation 207 transmits the data of the ultrasonic image 240, the data of the specimen identification information 42, the data of the examination result 43 of the specimen 90, etc., to the host computer 3 via the network 4.

### (Association of Ultrasonic Image and Specimen Identification Information)

In the second embodiment, in the same manner as in the control unit 60 of the first embodiment, the control unit 207a is configured to associate the specimen 90 sampled from the subject T with the ultrasonic image 240 in which the sampling position P of the specimen 90 is distinguishably displayed, by the specimen identification information 42. Specifically, the control unit acquires the data of the identification information 5a and the data of the examination result 43 of the specimen 90 from the examination result input device 2 via the communication unit 207f. Further, the control unit 207a acquires the data of the identification information 5a and the ultrasonic image 240 capable of identifying the sampling position P from the ultrasonic imaging device 201.

Then, the control unit 207a associates the specimen identification information 42 with the ultrasonic image 240 capable of identifying the sampling position P. That is, the control unit 60 associates the sampling position identifying information 44 and the examination result 43 of the specimen 90 with the ultrasonic image 240 capable of identifying the sampling position P via the identification information 5a. By performing the association operation in the workstation 207, it is possible to perform the association operation at a position (position where the workstation 207 is installed) which is away from a place such as a treatment room where the specimen 90 is sampled. Since the other configuration of the diagnostic imaging system 200 of the second embodiment is similar to the diagnostic imaging system 100 of the first embodiment described above, its description will be omitted.

As a flow of the association process, the processes of Steps S1 to S4 shown in FIG. 6 are performed in the ultrasonic imaging device 201, and the processes in Steps S5 and S6 shown in FIG. 6 are performed in the workstation 207.

### (Effects of Second Embodiment)

In the second embodiment, the following effects can be obtained.

In the second embodiment, as described above, the ultrasonic image 240 capable of identifying the sampling position P at the time of sampling the specimen 90 from the subject T is associated with the specimen identification information 42 sampled from the subject T. With this, in the same manner as in the first embodiment, it is possible to reduce the burden of a doctor, etc., involved in handling the sampled specimen 90.

In the second embodiment, as described above, when the specimen 90 of the sampling position P is sampled from the outside of the subject T, the puncture needle 214 is imaged in the ultrasonic image 240 by the ultrasonic transducer 215. Accordingly, when identifying the sampling position P based on the image of the puncture needle 214 included in the ultrasonic image 240, it is possible to easily identify the sampling position P from the position of the puncture needle 214. Further, in the ultrasonic image 240 captured from the outside of the subject T by the ultrasonic transducer 215, unlike the case of using the ultrasonic image 240 by the ultrasonic transducer which is placed inside the subject T and the imaging position cannot be visually recognized directly, the imaging position at the subject T can be easily identified. Therefore, it is possible to accurately identify the sampling position P in the subject T. Note that the other effects of the second embodiment are the same as those of the first embodiment.

### Modified Embodiment

It should be understood that the embodiments disclosed here are examples in all respects and are not restrictive. The scope of the present invention is shown by the scope of the claims rather than the descriptions of the embodiments described above, and includes all changes (modifications) within the meaning of equivalent and the scope of claims.

For example, in the first embodiment, an example in which the diagnostic imaging system 100 is configured by the ultrasonic endoscope device 1, the examination result input device 2, and the host computer 3 is described. Further, in the first embodiment described above, an example in which the diagnostic imaging system 200 is configured by the ultrasonic imaging device 201, the examination result input device 2, the host computer 3, and the workstation 207, but the present invention is not limited thereto. In the present invention, the system configuration of the diagnostic imaging system is not limited to the aforementioned embodiment, and, for example, a host computer may not be provided.

In the above-described first and second embodiments, an example in which a doctor, etc., is input the examination result 43 to the examination result input device 2 is shown, but the present invention is not limited to this. In the present invention, in cases where the specimen is, for example, blood and body fluids that can be analyzed for ingredients, a sample analyzer that automatically analyzes a specimen may be used instead of the examination result input device. In this case, the analysis result of the sample analyzer is used as the specimen identification information.

Further, in the first and second embodiments, an example in which the tip of the puncture needle 14 (214, specimen device) is set to the sampling position P is described, but the present invention is not limited thereto. In the present invention, in cases where it is difficult to confirm the tip of the puncture needle, an ultrasonic image capable of identifying the sampling position may be identified at another position of the puncture needle which is considered to be located near the sampling position.

Further, in the first and second embodiments, an example in which the puncture needle 14 (214) is used as a specimen device is described, but the present invention is not limited thereto. In the present invention, any device other than a puncture needle may be used as a specimen device as long as a specimen can be sampled.

1. A diagnostic imaging system comprising:
an ultrasonic imaging unit configured to capture an ultrasonic image of a subject; and
a control unit configured to associate an ultrasonic image capable of identifying a sampling position at the time of sampling a specimen from the subject among ultrasonic images captured by the ultrasonic imaging unit with information for identifying the specimen sampled from the subject.

2. The diagnostic imaging system as cited in claim 1,
wherein the information for identifying the specimen includes information for identifying the sampling position, and
wherein the information for identifying the sampling position is associated with the ultrasonic image at the time of sampling the specimen.

3. The diagnostic imaging system as cited in claim 1,
wherein the ultrasonic image capable of identifying the sampling position includes an ultrasonic image capable of identifying the sampling position by an image of a specimen sampling device arranged at the sampling position of the specimen or near the sampling position of the specimen.

4. The diagnostic imaging system as cited in claim 3,
wherein the specimen sampling device includes an introduction portion to be introduced into the subject and a sampling portion provided at a tip end portion of the introduction portion and configured to sample the specimen at the sampling position, and
wherein the ultrasonic imaging unit includes an internal ultrasonic transducer provided at the tip end portion of the introduction portion together with the sampling portion of the specimen sampling device and configured to capture the ultrasonic image of the subject by generating ultrasonic waves in the subject and detecting reflected waves.

5. The diagnostic imaging system as cited in claim 4,
wherein the sampling portion is arranged in the vicinity of the internal ultrasonic transducer and is arranged to be displayed in the ultrasonic image captured by the internal ultrasonic transducer.

6. The diagnostic imaging system as cited in claim 3,
wherein the ultrasonic imaging unit includes an external ultrasonic transducer configured to capture the ultrasonic image of the subject by generating ultrasonic waves from an outside of the subject and detecting reflected waves, and
wherein when the specimen at the sampling position is sampled from the outside of the subject, the specimen sampling device is imaged in the ultrasonic image by the external ultrasonic transducer.

7. The diagnostic imaging system as cited in claim 1,
wherein the information for identifying the specimen includes information for identifying the sampling position and identification information assigned for each specimen at the time of sampling.

8. The diagnostic imaging system as cited in claim 1,
wherein the information for identifying the specimen includes information for identifying the sampling position, and
wherein the control unit is configured to further associate information for identifying the subject with each of a plurality of ultrasonic images associated with the information for identifying the sampling position.

9. The diagnostic imaging system as cited in claim 1,
wherein the information for identifying the specimen includes information for identifying the sampling position and an examination result of the specimen sampled at the sampling position.
